(19) United States
(12) Patent Application Publication
(10) Pub. No.: US 2014/0316496 A1
(43) Pub. Date: Oct. 23, 2014

(54) Intravascular Electrode Arrays for Neuromodulation

(71) Applicant: NeuroTronik IP Holding (Jersey) Limited, St. Helier (JE)

(72) Inventors: Stephen C. Masson, Raleigh, NC (US); R. Frederick McCoy, JR., Chapel Hill, NC (US)

(73) Assignee: NeuroTronik IP Holding (Jersey) Limited, St. Helier (JE)

(21) Appl. No.: 14/084,829

(22) Filed: Nov. 20, 2013

Related U.S. Application Data
(60) Provisional application No. 61/728,806, filed on Nov. 21, 2012.

(51) Int. Cl. A61N 1/05

(52) U.S. Cl. CPC A61N 1/05
USPC 607/116

(57) ABSTRACT

A neuromodulation catheter positionable within a blood vessel for transvascular nerve stimulation includes a catheter body and an electrically insulative substrate carried at a distal end of the catheter body. A distal end of the substrate includes a plurality of laterally spaced-apart fingers. The substrate includes a first face and a second face on an opposite side of the substrate from the first face. A plurality of electrodes are disposed on the first face of the substrate such that each of the fingers has a plurality of the electrodes longitudinally spaced thereon. A support at the distal end of the catheter is expandable within the blood vessel to bias the first faces of the fingers against the wall of a blood vessel so as to bias at least a portion of the electrodes in contact with the wall.

This application claims the benefit of U.S. Provisional Application No. 61/728,806, filed Nov. 20, 2012, which is incorporated herein by reference.

### TECHNICAL FIELD OF THE INVENTION

The present application generally relates to intravascular electrode arrays for use in neuromodulation. More particularly, the application relates to electrode arrays and biasing supports used to position and bias the intravascular electrodes against the interior wall of a blood vessel.

### BACKGROUND

Prior applications filed by an entity engaged in joint research with the owner of the present application decribe neuromodulation methods using electrodes positioned in a blood vessel. The electrodes disposed inside the blood vessel are energized to stimulate or otherwise modulate nerve fibers or other nervous system targets located outside the blood vessel. Those prior applications include U.S. Publication No. 2007/0255379, entitled Intravascular Device for Neuromodulation, U.S. 2010/0023088, entitled System and Method for Transvascularly Stimulating Contents of the Carotid Sheath, U.S. application Ser. No. 13/281,399, entitled Intravascular Electrodes and Anchoring Devices for Transvascular Stimulation, International Application PCT/US12/35712, entitled Neuromodulation Systems and Methods for Treating Acute Heart Failure Syndromes, and U.S. application Ser. No. 13/547,031 entitled System and Method for Acute Neuromodulation, filed Jul. 11, 2012 (Attorney Docket: IAC-1260). Each of these applications is fully incorporated herein by reference. The latter application describes a system which may be used for hemodynamic control in the acute hospital care setting, by transvascularly directing therapeutic stimulus to parasympathetic nerves and/or sympathetic cardiac nerves using an electrode array positioned in the superior vena cava (SVC).

Proper placement of intravascular electrodes is essential for neuromodulation. The electrodes must be positioned to capture the target nerve fibers, while avoiding collateral stimulation of non-target nerve fibers. Mapping procedures are typically performed at the time of electrode placement to identify the optimal electrode location. Mapping can be manually controlled by the clinician or automatically controlled by the neuromodulation system. During mapping, different electrodes, combinations of electrodes, or arrays can be independently energized while the target response to the stimulus is monitored. For stimulation relating to cardiac or hemodynamic function, parameters such as heart rate, blood pressure, ventricular inotropy and/or cardiac output might be monitored. In some cases mapping includes additional steps of repositioning the electrode carrying member so as to allow additional electrode sites to be sampled. The mapping process is performed until the optimal electrode or combination of electrodes for the desired therapy array is identified.

The present application describes electrode support configurations that may be used in chronically-implantable or acute neuromodulation systems, including, but not limited to, those described in the referenced applications.

### BRIEF DESCRIPTION OF THE DRAWINGS

FIG. 1A is a top, cross-section view of the superior vena cava (SVC) illustrating a target electrode region for delivery of therapy to parasypathetic and sympathetic targets.

FIG. 1B is an anterior view of the SVC illustrating the target region depicted in FIG. 1A.

FIG. 2 is a plan view illustrating a first embodiment of an electrode array.

FIGS. 3, 4A and 4B are plan views illustrating second, third, and fourth embodiments, respectively, of electrode arrays.

FIGS. 5, 6 and 7 are elevation views of first, second and third support structures on catheter bodies. The support structures are schematically shown disposed within a blood vessel with the portion of the support structure that carries the electrode arrays (not shown) biased against the wall of the blood vessel.

FIG. 8 illustrates positioning of a support stucture in a blood vessel within which cardiac rhythm management leads have been previously positioned.

### DETAILED DESCRIPTION

This application describes intravascular electrode arrays and associated supports used to bias neuromodulation electrodes against the wall of a blood vessel. In general, the electrode arrays and associate supports may be elements of a catheter that includes a catheter body, the support structure on a distal portion of the catheter body, and the electrode array on the support structure. As disclosed in the prior applications, electrodes in the electrode array are electrically coupled to a neurostimulator that energizes the electrodes using stimulation parameters selected to capture the target nerve fibers and to achieve the desired patient effect.

The illustrated electrode supports are designed to bias arrays of multiple electrodes in contact with the surrounding vascular wall—such that when energy from an associated neuromodulation system energizes the electrodes, target nerve fibers outside the blood vessel are captured. The embodiments are designed to position the electrodes in positions suitable for delivering electrical therapy to the target fibers from the intended position of the array within the vasculature. The disclosed embodiments also give the user (or the automated mapping feature of a neuromodulation system) a variety of electrodes to select between when choosing the optimal electrode or electrode combination to deliver the intended therapy.

For convenience this description focuses on the use of the described electrodes and support structures in a system used to deliver electrical therapy to parasympathetic and sympathetic nerve fibers using electrodes on a single electrode carrying member positioned in the SVC, e.g. in accordance with systems and methods of the type disclosed in U.S. application Ser. No. 13/547,031 entitled System and Method for Acute Neuromodulation, filed Jul. 11, 2012. However, the disclosed concepts are equally suitable for use in other clinical applications, including those that deliver stimulus from electrodes disposed within other vessels and those where electrodes on the electrode carrying member deliver electrical therapy to only a single type of nerve fiber.

### Electrode Arrays

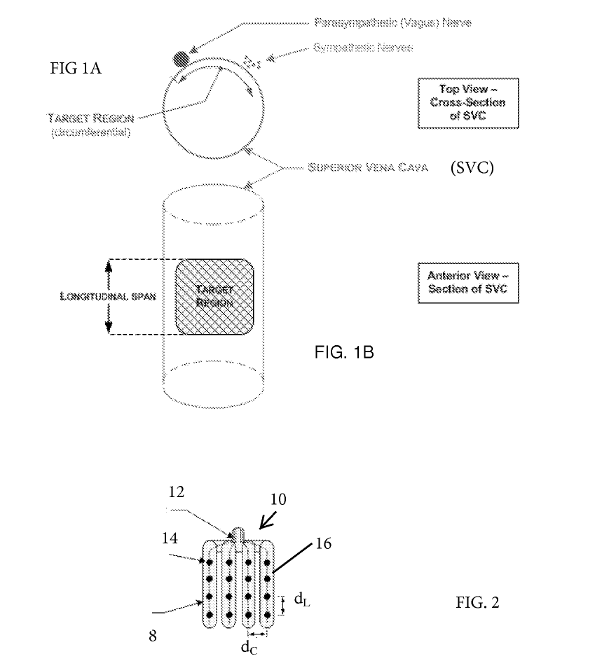

The exemplary electrode arrays may be positioned on the distal portion of an intravascular catheter (also referred to herein as a “neurocatheter”). For hemodynamic control of the type disclosed in U.S. application Ser. No. 13/547,031, an optimal electrode array places electrodes against the SVC wall in order to transvascularly stimulate parasympathetic and/or sympathetic cardiac nerves. Prior studies have identified areas on the posterior wall of the mid-to-cranial SVC, between the brachiocephalic junction and right atrium, where both parasympathetic and sympathetic nerves can be electrically stimulated. The use of an array of electrodes on the catheter allows general placement into a target region of the SVC without a requirement for precise placement. Once the electrode array is placed into this general SVC target region, mapping can be performed by the neuromodulation system or user to determine which electrodes in the array achieve optimal results. This target region can be defined by both a longitudinal range of the SVC, and by a circumferential range of the SVC (see FIGS. 1A and 1B). Previous disclosures have identified the preferred longitudinal range as the mid-to-cranial SVC, and preferred circumferential range along the posterior side of the SVC.

It is known that accessing the human SVC using the widely accepted, standard percutaneous procedure, especially from venous access sites such as the internal jugular, subclavian or femoral veins is a simple and straightforward technique, in which a variety of clinicians are proficient. In order to provide for both ease-of-use in the acute hospital setting and allow for positioning without the use of imaging, such as fluoroscopy, the NC contains an “array” of electrodes to provide a coverage area for capture of target cardiac nerves. All of the electrodes in the array can then be connected to the neuromodulation system, which can then “select” the desired anodes and cathodes by means of electronic switching circuitry in its response mapping function.

In a preferred arrangement, the electrode array includes a flexible substrate. The substrate is preferably formed of an insulating material, such as a polymer (including silicone, polyurethanes, polyimide, and copolymers) or a plastic. Thus electrode surfaces will be exposed on one side of the array (the side intended to be against the SVC wall) and insulated by the substrate on the other side of the array in order to capture target nerves through the SVC wall with efficient stimulation energies, and avoid collateral stimulation through the blood pool. Where the neurocatheter is to be used for acute use (typically 36-72 hours, but in general less than 7 days), the electrodes may be constructed of a variety of alloys, including stainless steel, titanium, cobalt chromium, and platinum alloys.

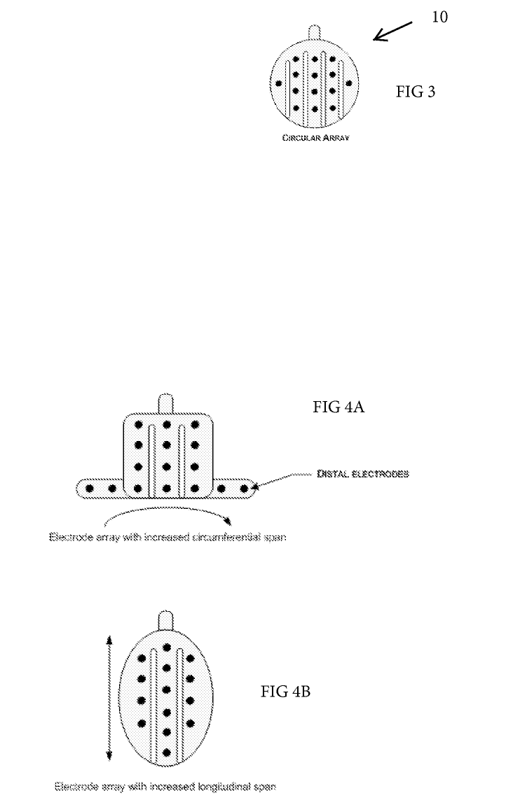

The electrodes are arranged on the substrate in a variety of geometries in order to provide the desired stimulation “coverage region” (both circumferentially and longitudinally). FIG. 2 shows a preferred electrode array on substrate 8. The array is arranged in a rectangular shape that contains a 4×4 array of electrodes 14 for a total of 16 electrodes. The drawing shows what would be the posterior face of the electrode array within the SVC (i.e. the face that contacts the posterior region of the wall of the SVC lumen). In this arrangement, the substrate has a geometry resembling a fork—with a plurality of parallel, longitudinally extending tines or fingers 16 laterally separated from one another at their distal ends. Linear arrangements of electrodes are disposed on each such finger 16. Other preferred embodiments include other geometric arrays that contain from 4 to 32 electrodes, and that can be arranged in, or on substrates having, rectangular, circular (FIG. 3), oval (FIG. 4B) or irregular configurations, such as the one shown in FIG. 4A. As shown, these embodiments can likewise include longitudinally-extending fingers 16 with linear arrangements of electrodes disposed on them. These can be arranged to provide an effective coverage area that spans greater circumferential area as depicted in FIG. 4A, or greater longitudinal area, as depicted in FIG. 4B.

Independent of geometric shape, each electrode in the array will be spaced from adjacent electrodes by a longitudinal distance, d_{L}, and a circumferential distance, d_{C}. The spacing between electrodes is chosen to optimize capture of target nerves, and may be from 1 to 10 mm, typically 5 mm, and the longitudinal and circumferential spacing may be equal or may differ.

In some embodiments, the array might include a greater circumferential expanse of electrodes in the distal electrodes (see, e.g. FIG. 4A), which in use are positioned closest to the right atrium. Where the neurocatheter is introduced using a femoral approach, the most proximal electrodes in the array will lie closest to the atrium and might be provided with a greater circumferential expanse. This arrangement can facilitate positioning methodologies that allow safe positioning of the array so as to avoid the risk of atrial capture, as disclosed in co-pending U.S. application Ser. No. 14/______, entitled Positioning Methods for Intravascular Electrode Arrays for Transvascular Neuromodulation, filed Nov. 20, 2013 (Attorney Docket NTK-220), which is incorporated herein by reference.

The electrodes can be constructed on the substrate using a variety of manufacturing techniques, including subtractive manufacturing processes (such as mechanical removal by machining or laser cutting), additive processes (such as laser sintering, deposition processes, conductor overmolding), or combinations (such as printed circuit technology with additive plating). When assembled on the catheter, the electrodes and substrate (where used) will be attached to or manufactured on a mechanical support structure (described below) having features for biasing the electrodes against the vascular wall and, optionally, supporting the distal end of the neurocatheter against the vascular wall or spaced from the vascular wall.

### Mechanical Support Structure for the Electrode Array

In order to capture target nerves through the SVC wall with efficient stimulation energies, secure engagement of the electrodes against the SVC wall is desired. Therefore, the catheter includes a support structure or structures that provide mechanical force to press the electrode surfaces against the SVC wall once deployed. Additionally, the support structure securely but reversibly (at least in the case of an acute device) anchors the catheter to prevent its migration within the vasculature. The support structures are constructed of a variety of shape memory alloys, such as nickel-titanium, or other alloys that would be mechanically positioned by mechanisms in the catheter body. Where the substrate described above is used to form the array, the support structures may be integral with the substrate, or coupled to the substrate.

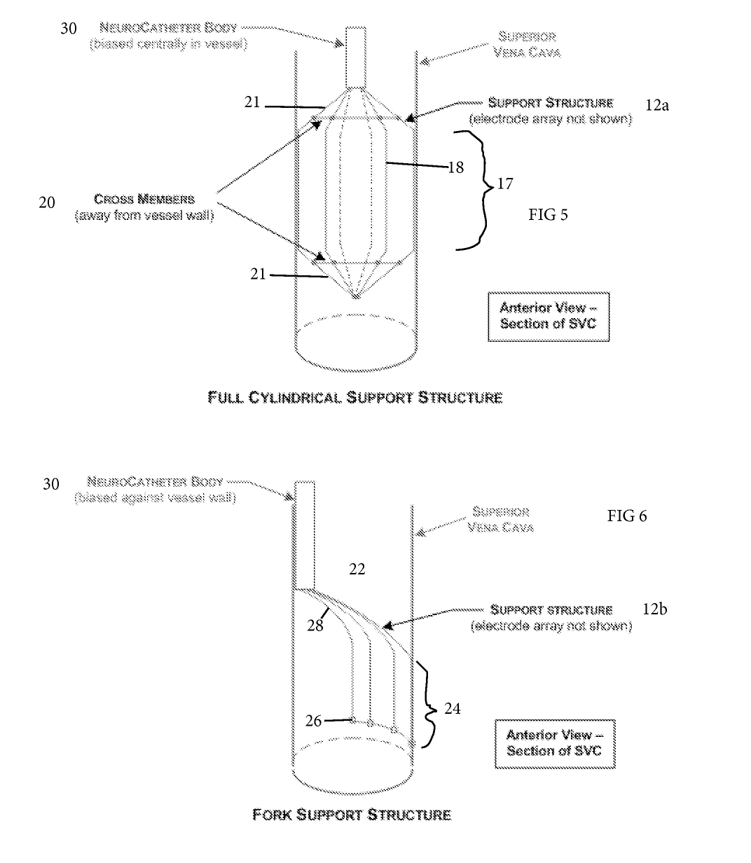

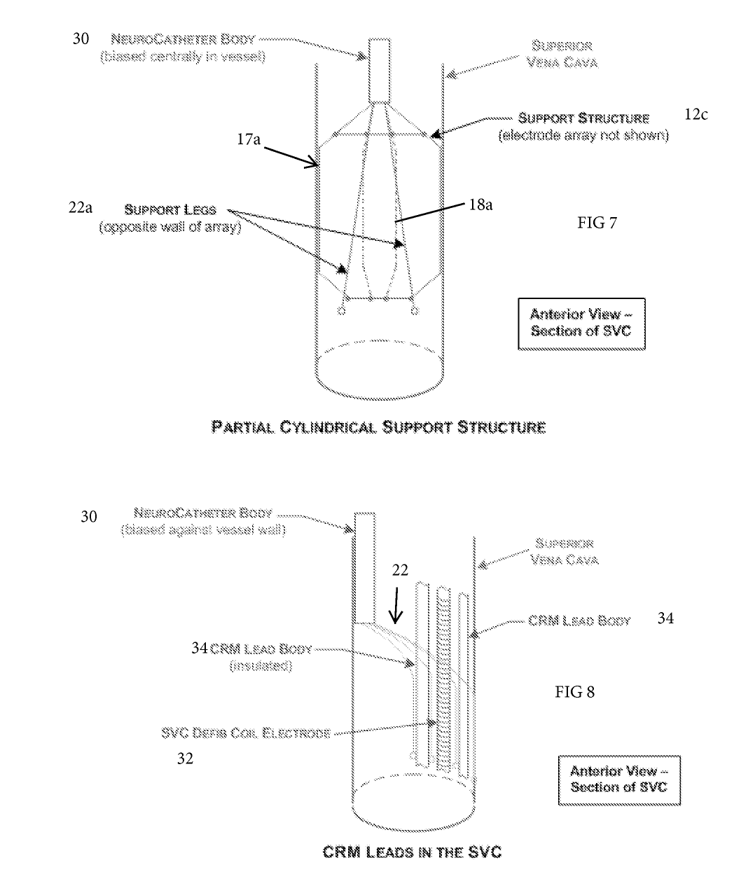

Preferred embodiments for the support structures include a full cylindrical configuration, shown in FIG. 5, a fork configuration, shown in FIG. 6, and a partial cylindrical configuration, shown in FIG. 7. In all of these configurations, the electrode array, which is only required to cover a portion of the circumference of the SVC, will be attached to the support structure. In FIGS. 5 through 7, the electrode array is not shown to allow the support structure to be more easily seen. In preferred embodiments, the electrode arrays with the associated insulative substrates disclosed above are used.

In use, the support structure is radially expanded at the target electrode site within the vasculature using known means. For example, the support structure with the attached electrode array may be compressed within a deployment sheath for advancement through the vasculature, and then released from the deployment sheath at the target electrode site. The support structure self expands at the target site, or is actively expanded using a balloon or other expansion structure, positioning and biasing the electrodes against the vessel wall.

The full cylindrical support structure 12a (FIG. 5) includes a cylindrical portion 17 formed of a plurality of parallel longitudinally-extending support elements 18. When the support structure is expanded within the vasculature, the support elements of the cylindrical portion contact the vessel wall. The electrode array is carried on the cylindrical portion of the support structure, and in particular is mounted to a subset of the longitudinally-extending support elements that expands towards the target SVC wall region. For example, each of the longitudinal fingers 8 of the substrate shown in FIG. 2 may extend along four adjacent longitudinally-extending elements 18 of the cylindrical part of the support structure. The opposed longitudinally-extending support elements forming the cylindrical portion contact the opposed portion of the vessel wall, such that the entire array of longitudinal support elements aid in securing the electrode array to the vessel wall.

In the FIG. 5 arrangement, the support structure biases the distal end of the neurocatheter's body 30 towards the center of the SVC lumen as shown. This central positioning of the neurocatheter body 30 and deployed structure for the electrode array creates a uniform shape that results in central, more uniform laminar blood flow patterns to prevent thrombosis. In this central design, adequate space does exist through openings in the support structure on either side of the catheter body to allow other catheters or devices to be inserted through the SVC when needed. The FIG. 5 embodiment includes cross members 20 to provide circumferential structure. These cross members are positioned at the distal and, optionally, proximal ends of the support structure between struts 21 that angle inwardly from the longitudinally-extending members. This places the cross members struts away from the vessel wall when the structure is fully deployed as shown in FIG. 5, rather than between the longitudinally-extending members of the cylindrical portion, thus leaving the spaces between the longitudinally-extending members 18 (and thus the spaces between the longitudinal columns of electrodes on those members) clear. Where the electrodes are being deployed in an area of the SVC where cardiac rhythm management (CRM) leads reside, these spaces give room for the columns of electrodes on the neurocatheter to circumferentially shift around existing lead bodies and into contact with an adjacent portion of the vessel wall, as will be discussed in further detail in connection with FIG. 8.

Another embodiment of a support structure is the fork support structure 12b shown in FIG. 6. This embodiment includes a series of longitudinal members 22. These members have distal sections 24 running in parallel to one another and including free distal ends 26, similar to tines of a fork. Proximal sections 28 extend from the neurocatheter body 30 to the proximal ends of the distal sections 24. The electrodes are positioned along the distal sections (with or without the substrate arrangements described above). When released from a deployment sheath, the fork structure expands to position each of the distal sections 24 and their associated electrodes against the SVC wall. This arrangement minimizes material and facilitates better compression into the deployment sheath to minimize delivery diameter. By applying mechanical forces on one side of the wall (in contrast with the forces applied around the cylinder in the FIG. 5 embodiment), the distal portion of the neurocatheter body 30 is biased towards the portion of the SVC wall opposite to the portion against which the electrodes are biased, as shown in FIG. 6. This leaves maximum cross-sectional space between the longitudinal members, allowing other catheters or devices to be inserted through the SVC, as would be typically required for patients in acute hospital care. In addition, by having the neurocatheter body positioned against the vessel wall, central blood flow disruption can be minimized to prevent thrombosis. Also, like the full cylindrical structure and as discussed further in connection with FIG. 8, the individual longitudinal members 22 leave space between the longitudinal columns of electrodes so that if, upon expansion of the support structure, the longitudinal columns collide with resident CRM leads within the SVC, the columns can shift into contact with SVC wall space adjacent to the CRM leads.

The support structure 12c of the FIG. 7 embodiment combines features of the FIGS. 5 and 6 embodiments by combining a partially cylindrical structure with opposing mechanical support legs 22a (e.g. two or more legs of the type used in the FIG. 6 embodiment). In this configuration, a partial cylinder 17a of support elements 18a carrying the electrode array is secured against the SVC wall on one side of the deployed support structure, and a number of opposing elements or legs 22a expands on the other side of the support structure to provide an equal and counteracting force against the SVC wall opposite the target region. The partial cylindrical structure may incorporate features of the FIG. 5 support structure, but will extend less than 360 degrees around the circumference of the SVC. The number of opposing legs 22a would preferably be 2, but more legs, up to 6, can be included. In this arrangement the distal portion of the neurocatheter body 30 is again biased towards the center of the SVC, as in the full cylindrical configuration. In a variation of this embodiment, the support legs 22a shown in FIG. 7 may be positioned against the blood vessel wall on one side of the blood vessel, and the members 24 of the fork-like structure of the FIG. 6 embodiment (with the electrode array thereon) positioned on the opposed portion of the blood vessel wall.

### NeuroCatheter Use in the Presence of Existing CRM Leads

As noted above, in some cases the neurocatheter may be used in patients having permanently implanted CRM devices and the chronic leads that are used with such devices. Such CRM leads typically run from the transvenous entry site of the subclavian vein through the SVC towards the heart. As a result, some patients will have leads existent in the SVC when the neurocatheter is deployed. Also, it is conceivable that one or more lead bodies will lie in the target region for parasympathetic and sympathetic nerve capture. The CRM lead bodies 34, which are covered with silicone or polyurethane insulation, may be free floating in the vessel or attached to the vessel wall and covered with fibrotic or scar tissue (either partially or fully covered), as shown in FIG. 8. Also, in the case of defibrillators and cardiac resynchronization therapy defibrillators, a conductive defibrillation coil electrode 32 may be existent, also shown in FIG. 8. As a result, the neurocatheter's electrodes may encounter lead insulation, scar tissue or fibrosis, or the conductive defibrillation coil.

Features of the disclosed electrode array allow target nerve capture despite the presence of CRM leads. In particular, the mechanical layout and design of the neurocatheter electrode array and support structure facilitate engagement in the presence of CRM lead bodies. A critical and common feature of both the fork and cylinder support structures is that they have parallel elements with openings where engaged to the target SVC vessel wall. These openings provide the most flexibility when engaging against the vessel wall in the presence of chronic CRM leads, by allowing the electrodes to engage against irregular surfaces presented by attached lead bodies and the ability to have the longitudinal electrodes engage the SVC wall by moving between or around free floating leads to engage active tissue.

It should be recognized that a number of variations of the above-identified embodiments will be obvious to one of ordinary skill in the art in view of the foregoing description. Moreover, it is contemplated that aspects of the various disclosed embodiments may be combined to produce further embodiments. Accordingly, the invention is not to be limited by those specific embodiments and methods of the present invention shown and described herein. Rather, the scope of the invention is to be defined by the following claims and their equivalents.

All prior patents and applications referred to herein, including for purposes of priority, are incorporated by reference for all purposes.

What is claimed is:

1. A neuromodulation catheter positionable in a blood vessel having a wall, comprising:
a catheter body;
an electrically insulative substrate carried at a distal end of the catheter body, a distal end of the substrate including a plurality of laterally spaced-apart fingers, the substrate having a first face and a second face on an opposite side of the substrate from the first face;
a plurality of electrodes on the first face of the substrate, each of the fingers having a plurality of said electrodes longitudinally spaced thereon; and
a support at the distal end of the catheter, the support expandable within the blood vessel to bias the first faces of the fingers against the wall of a blood vessel so as to bias at least a portion of the electrodes in contact with the wall.

2. The catheter of claim 1, wherein the support is further expandable to bias a distal section of the catheter body against the wall of the blood vessel.

3. The catheter of claim 1, wherein the support is further expandable to support a distal section of the catheter body within the lumen of the blood vessel, spaced apart from the wall.

4. The catheter of claim 1, wherein the support includes a plurality of spaced-apart members extending distally from the catheter body, wherein each finger is positioned on a different one of said spaced-apart members.

5. The catheter of claim 4, wherein each member includes a proximal portion extending from the catheter body, and a distal portion extending angularly from the proximal portion, wherein the distal portions of the longitudinal members are parallel to one another.

6. The catheter of claim 4, wherein each longitudinal member includes a free distal end.

7. The catheter of claim 1, wherein the substrate is formed to have a generally rectangular planar shape, wherein the fingers comprise the rectangular shape.

8. The catheter of claim 1, wherein the substrate is formed to have a generally circular or oval planar shape, wherein the fingers comprise the circular or oval shape.

9. The catheter of claim 1, further including a pair of lateral fingers each extending from one of the fingers, the lateral fingers including electrodes thereon.

10. A neuromodulation catheter positionable within a blood vessel having a wall, comprising:
a catheter body;
a support at the distal end of the catheter body;
a plurality of electrodes carried by the support, the support expandable within the blood vessel to bias a distal portion of the catheter body in contact with the blood vessel wall, and to further bias the electrodes against a portion of the blood vessel wall that is circumferentially offset from the distal portion of the catheter body.

11. The neuromodulation catheter of claim 10, wherein the support is positioned such that when a distal portion of the catheter body is positioned in contact with the blood vessel wall, the support biases the electrodes against a portion of the blood vessel wall that is longitudinally offset from the distal portion of the catheter body.

12. The neuromodulation catheter of claim 10, wherein the support includes a plurality of elements, each element having a proximal portion and a distal portion, wherein the distal portions of the elements extend parallel to one another, and wherein each distal portion includes a free distal end.

13. A neuromodulation catheter positionable within a blood vessel having a wall, comprising:
a catheter body;
a support at the distal end of the catheter body;
a plurality of electrodes carried by the support, the support expandable within the blood vessel to support a distal end of the catheter body within the blood vessel lumen offset from the wall, and to bias the electrodes against a portion of the blood vessel wall.

14. The neuromodulation catheter of claim 13, wherein the support includes a cylindrical portion comprising a plurality of longitudinal members extending parallel to one another, wherein the electrodes are positioned on at least two of the longitudinal members.

15. The neuromodulation catheter of claim 13, wherein the support includes:
a partially cylindrical portion comprising a plurality of longitudinal members extending parallel to one another, wherein the electrodes are positioned on at least two of the longitudinal members, the partially cylindrical portion expandable into contact with a portion of the blood vessel wall; and
at least one member expandable into contact with a portion of the blood vessel wall opposite to the partially cylindrical portion.
